(19) United States
(12) Patent Application Publication
(10) Pub. No.: US 2009/0104288 A1
(43) Pub. Date: Apr. 23, 2009

(54) Compositions and methods for inhibiting a honey bee pathogen infection or controlling a hive infestation

(75) Inventor: Gene Probasco, Yakima, WA (US)

Correspondence Address: EDWARDS ANGELL PALMER & DODGE LLP, P.O. BOX 55874, BOSTON, MA, 02205, US

(73) Assignee: John I. Haas, Inc., Washington, DC (US)

(21) Appl. No.: 11/985,568

(22) Filed: Nov. 15, 2007

Related U.S. Application Data
(60) Provisional application No. 60/859,373, filed on Nov. 15, 2006.

(51) Int. Cl. A61K 36/185; A01K 51/00; A01K 47/00; A61K 31/19

(52) U.S. Cl. 424/725; 449/2; 449/3; 514/557

(57) ABSTRACT

The present invention is directed to methods and compositions to prevent or treat a honey bee pathogen infection (e.g., Melissococcus plutonis, Paenibacillus larvae, Ascosphaera apis). Specifically, the invention provides for the treatment or prevention of European or American foulbrood or chalkbrood. In addition, the invention provides methods for controlling Varroa mites that can weaken a hive or act as vectors for bacterial diseases. In further embodiments, the invention provides for the treatment or prevention of hive infestations with Lepidopteran pests, such as the wax moth (Galleria mellonella).

### CROSS-REFERENCE TO RELATED APPLICATION

This application claims the benefit of U.S. Provisional Application No. 60/859,373, filed on Nov. 15, 2006, the entire contents of which are incorporated herein by reference.

### BACKGROUND OF THE INVENTION

Honey bees, Apis mellifera, are required for the effective pollination of crops and are therefore critical to world agriculture. Honey bees also produce economically important products, including honey and bee's wax. Honey bees are susceptible to a number of parasites, including the ectoparasitic mite, Varroa destructor. Varroa mites parasitize pupae and adult bees, using their mouths to puncture the honey bee's exoskeleton and feed on the bee's hemolymph. These wound sites often harbor pathogen infections. Honey bee pathogens include the bacterial pathogens Melissococcus plutonis, which causes European foulbrood, and Paenibacillus larvae, which causes American foulbrood. Honey bees are also susceptible to fungal pathogens, such as Ascosphaera apis, which causes chalkbrood.

Varroa mites are suspected to act as vectors for a number of honey bee pathogens, and may weaken the immune systems of their hosts, leaving them vulnerable to infections. Weakened bee colonies are also susceptible to infestations by insect pests, such as the wax moth, Galleria mellonella. If left untreated honey bee pathogens, alone or in combination with Varroa mites and wax moths, may weaken or even destroy the hive. Maintaining a supply of strong honey bee colonies available for pollination is essential for the sustained production of farm crops worth more than $14 billion to U.S. agriculture. American foulbrood, European foulbrood, chalkbrood, and wax moth infestations, either alone or in combination with Varroa mite parasites, have devastating effects on bee biology and the bee's ability to pollinate that are reflected in losses in production of agricultural crops and honey bee products. Increased drug resistance by pathogenic microbes and insect pests has created an urgent demand for introduction of new biocides that kill or inhibit bacterial and/or fungal pathogens, and that control destructive insect or acarid infestations of honey bee hives.

### SUMMARY OF THE INVENTION

As described below, the present invention features methods and compositions for preventing or treating a honey bee pathogen infection or hive infestation.

In one aspect, the invention provides a method of inhibiting a honey bee pathogen. The method involves contacting the pathogen with an effective amount of a composition comprising a hop derivative, thereby controlling a honey bee pathogen.

In another aspect, the invention provides a method of treating or preventing a pathogen infection in a honey bee hive. The method involves contacting the hive with an effective amount of a composition comprising a hop derivative, thereby treating or preventing a pathogen infection in a honey bee hive.

In another aspect, the hive is identified as in need of treatment with an effective amount of a hop derivative or a composition comprising a hop derivative.

In yet another aspect, the invention provides a method of treating or preventing a destructive insect (e.g., Lepidoptera, such as the wax moth, Galleria mellonella, or small hive beetle) infestation in a honey bee hive. The method involves contacting the hive with an effective amount of a composition comprising a hop derivative, thereby treating or preventing a Lepidoptera infestation in a honey bee hive. In one embodiment, the method treats galleriasis.

In yet another aspect, the invention provides a method for maintaining the health of a honey bee hive. The method involves contacting the hive with an effective amount of a composition comprising a hop derivative, thereby maintaining the health of the honey bee hive. In one embodiment, the hive is maintained in an essentially pathogen-free state or essentially infestation-free state.

In another aspect, the invention provides a composition for treating or preventing a honey bee pathogen infection and/or hive infestation, the composition comprising an effective amount of a hop derivative in a suitable form for delivery to a bee or hive. In one embodiment, the effective amount inhibits the growth, proliferation, or survival of a bacterial or fungal pathogen. In another embodiment, the effective amount controls an acarid or destructive insect (e.g., Lepidoptera or small hive beetle). In one embodiment, the hop derivative is an alpha acid, beta acid, or combination of an alpha and a beta acid. In another embodiment, the composition comprises at least about 1000 ppm, 0.01%, 0.1%, 1%, 5%, 7%, 10%, 12%, 15%, 20% alpha or beta acids. In another embodiment, the composition comprises at least about 0.01%, 0.1%, 0.5%, 1%, 2%, 2.5%, 3%, or 5% alpha acids. In yet another embodiment, the composition comprises at least about 7.5% alpha acids. In still another embodiment, the composition comprises at least about 0.01%, 0.1%, 0.5%, 1%, 2%, 2.5%, 3%, 5%, 10%, or 15% alpha acids. In yet another embodiment, the composition comprises at least about 2.5% beta acids. In another embodiment, the composition comprises at least about 5% beta acids. In another embodiment, the composition comprises a combination of alpha and beta acids, e.g., at least about 2.5% beta acid and at least about 7.5% alpha acids. In yet another embodiment, the form is selected from the group consisting of a liquid, a powder, an oil, an emulsion, a capsule, and a vapor. In yet another embodiment, the composition further comprises a carrier medium.

In another aspect, the invention provides a controlled release composition for treating or preventing a composition for treating or preventing a honey bee pathogen infection and/or hive infestation, the composition comprising an effective amount of a hop derivative in a suitable form for delivery to a honey bee or honey bee hive. In one embodiment, the effective amount inhibits the growth, proliferation, or survival of a bacterial or fungal pathogen. In another embodiment, the effective amount controls an acarid or destructive insect. In another embodiment, the hop derivative is released over the course of at least about 7, 10, 14, 21, 28, or 41 days. In yet another embodiment, the hop derivative is released over the course of at least about 1, 2, 3, 6, 9, or 12 months.

In various embodiments of the above aspects, the pathogen infection is a Melissococcus plutonis, Ascosphaera apis, or Paenibacillus larvae infection. In other embodiments of any of the above aspects, the method treats or prevents American foulbrood, European foulbrood, or chalkbrood. In various embodiments of any of the above aspects, the hive infestation is a Varroa mite, wax moth infestation, or small hive beetle infestation.

In another aspect, the invention provides a biocide delivery device, the device comprising a composition of any aspect delineated herein. In one embodiment, the device is selected from the group consisting of a strip, controlled release strip, tablet, reservoir, polymer disc, evaporation device, fiber, tube, polymeric block, membrane, pellet, a packet, patty, and microcapillary.

In a related aspect, the invention provides a hive comprising a composition of any aspect delineated herein.

In another related aspect, the invention provides a honey bee product (e.g., honey, honey comb, or bees wax) produced in the hive of an aspect delineated herein.

In another aspect, the invention provides a kit for the treatment or prevention of a pathogen infection or hive infestation, the kit comprising an effective amount of a hop derivative in a form suitable for delivery to a site of infection or infestation. In one embodiment, the site of infection or infestation is a bee hive. In another embodiment, the site of infestation is a bee.

In another aspect, the invention provides a method of identifying a hop derivative that inhibits a bacterial or fungal pathogen. The method involves contacting a bacterial or fungal cell or culture with a test composition comprising a hop derivative; and assaying bacterial or fungal growth. In one embodiment, the assay is an agar diffusion assay. In one embodiment, the test compound inhibits Melissococcus plutonis, Paenibacillus larvae, or Ascosphaera apis growth. In another embodiment, the test compound kills Melissococcus plutonis, Paenibacillus larvae, or Ascosphaera apis. In another embodiment, the test compound kills bacterial or fungal spores. In another embodiment, the method further involves the steps of contacting a honey bee with the test composition; and assaying a honey bee biological function. In another embodiment, the method identifies a test compound that does not disrupt a honey bee biological function. In yet another embodiment, the method identifies a test compound that disrupts a honey bee biological function. In yet another embodiment, the test compound kills a honey bee.

In a related aspect, the invention provides a method of identifying a hop derivative that disrupts a biological function of a destructive insect (e.g., wax moth or hive beetle). The method involves contacting the destructive insect with a test composition comprising a hop derivative; and assaying the destructive insect's biological function. In one embodiment, the test compound disrupts a destructive insect biological function. In another embodiment, the test compound kills the adult destructive insect, larvae, or eggs. In another embodiment, the test compound reduces destructive insect reproduction. In another embodiment, the method further involves the steps of contacting a honey bee with the test composition; and assaying a honey bee biological function. In one embodiment, the method identifies a test compound that does not disrupt a honey bee biological function. In another embodiment, the method identifies a test compound that disrupts a honey bee biological function. In another embodiment, the test compound kills a honey bee.

In various embodiments of any of the above aspects, the pathogen is a bacterial pathogen (e.g., Melissococcus plutonis or Paenibacillus larvae) or fungal pathogen (e.g., Ascosphaera apis). In other embodiments of any of the above aspects, the pathogen infection is a bacterial infection, such as American or European foulbrood. In other embodiments of any of the above aspects, the pathogen infection is a fungal pathogen, such as Ascosphaera apis. In various embodiments of any of the above aspects, the hop derivative is an alpha acid (e.g., isoalpha acid, tetrahydroisoalpha acids, rho isoalpha acids), beta acid, or a combination of alpha and beta acids. In various embodiments, the composition comprises at least about 3, 5, 10, 15, 20, 25, 50, 75, 100, 200, 300, 400, 500, 1000, 1500 ppm alpha acids and/or beta acids. In particular embodiments of the above aspects, the composition comprises sodium (Na) or potassium (K) salts of beta acids. In various embodiments of any of the above aspects, the composition comprises at least about 0.01%, 0.1%, 1%, 5%, 6%, 7%, 8%, 9%, 10%, 12%, 15%, 20% alpha and/or beta acids. In various embodiments, contact with a hops derivative inhibits the growth or proliferation of a bacteria or fungus. In other embodiments, the contact with a hops derivative kills the bacteria or fungus. In various embodiments, contact with a hops derivative inhibits the growth, reproduction, or other biological function of a destructive insect. In other embodiments, contact with a hops derivative kills or repels the destructive insect (e.g., wax moth or small hive beetle).

In yet another aspect, the invention provides the use of a hop derivative or a composition comprising a hop derivative alone or together with one or more second therapeutic agents in the manufacture of a medicament, either as a single composition or as separate dosage forms, for treatment or prevention of a pathogen (e.g., honeybee pathogen) infection, including in a honey bee hive. Another aspect of the invention is a hop derivative or a composition comprising a hop derivative herein for use in the treatment or prevention of a pathogen (e.g., honeybee pathogen) infection, including in a honey bee hive.

Other features and advantages of the invention will be apparent from the detailed description, and from the claims.

### DEFINITIONS

By “acarid” is meant an arachnid of the order Acarina, which includes mites and ticks.

By “alpha acid” is meant an organic acid derived from a hop plant (Humulus lupulus) having structural homology to a humulone, adhumulone, cohumulone, or an analog or derivative thereof. Humulone, adhumulone, and cohumulone are the three most abundant alpha acid analogs. Other exemplary derivatives of an alpha acid include, but are not limited to isoalpha acids, rhoisoalpha acids, tetrahydroisoalpha acids, and hexahydroisoalpha acids.

By “bacterial pathogen” is meant any bacteria that disrupts the health or normal biological function of a honey bee or honey bee population.

By “beta acid” is meant an organic acid derived from a hop plant (Humulus lupulus) having structural homology to a lupulone, adlupulone, colupulone or an analog or derivative thereof. Lupulone, adlupulone, and colupulone are the three most abundant beta acid analogs. Other exemplary derivatives of a beta acid include, but are not limited to, hulupones, hexahydrobeta acids and hexahydro hulupones.

By “biocide” is meant an agent that slows, inhibits, or reduces the growth, proliferation, reproduction, or survival of a pathogen or destructive insect. Exemplary biocides are antibiotics, insecticides, and fungicides.

By “biological function” is meant any physiological or behavioral activity of an organism. Exemplary biological functions include reproduction, respiration, neural activity, locomotion. Honey production is a biological function that is specific to a honey bee.

By “contacting” is meant touching, associating with, or having proximity to a composition. For example, a hop derivative may contact a hive either inside or outside of the hive structure.

By “controlled release” is meant released over the course of hours, days, weeks, or months.

By “controlling” is meant inhibiting survival or reducing, slowing, or stabilizing the growth of a population (such as repelling a pest), or repelling a pest.

By “comb” is meant sections of hexagonal bee wax cells that are used to rear honey bee progeny (“brood”) and store honey and pollen.

By “destructive insect” is meant any insect capable of infesting and damaging the hive or honey bee population. The wax moth, Galleria mellonella, is one example of a destructive insect that increases larval death or causes damage to honey combs.

By “effective amount” is meant an amount effective to disrupt the biological function of a honey bee pathogen, acarid, or destructive insect. Biological functions include growth, proliferation, respiration, survival, reproduction or any other physiological function of an organism.

By “fungal pathogen” is meant any fungus that disrupts the health or normal biological function of a honey bee or honey bee population.

By “hive” is meant a structure that contains a bee colony. A modern box hive typically includes a bottom board, cover, and one or more boxes, stacked one above the other. Inside, each box contains a series of movable frames of comb or foundation held in a vertical position a bee space apart.

By “honey bee” is meant a Hymenopteran insect of the genus Apis. The term “honey bee” is not limited to the adult form of the insect, but encompasses all honey bee developmental stages, including but not limited to egg, larva, and pupa. Exemplary honey bee species include Apis mellifera and Apis cerana.

By “honey bee colony” is meant a community of bees. Honey bee colonies may occur in the wild or may be maintained by bee keepers.

By “maintain the health of the honey bee hive” is meant preserve the normal function of a hive. For example, the health of the hive is preserved where the honey bee population, as well as individual honey bees, is essentially free of pathogens or destructive insects or parasitizing acarids. A hive is “essentially free” of pathogen infection or infestation where the level of infection or infestation is sufficiently low as to be below the level of detection. In another embodiment, the level of infection or infestation is below the level sufficient to cause a 5% increase in larval death or a 5% reduction in the production of a honey bee product.

By “honey bee parasitic mite” is meant any acarid that parasitizes a honey bee. Exemplary honey bee parasitic mites include Varroa mites and tracheal mites.

By “honey bee pathogen” is meant any organism that interferes with the health or normal biological function of a honey bee. Exemplary honey pathogens include, but are not limited to, bacteria, fungus, viruses, and protozoa.

By “honey bee product” is meant any composition that is produced by honey bee activity. Exemplary honey bee products include, but are not limited to, honey, honey combs, and bee's wax.

By “hop derivative” is meant any molecule that naturally occurs in hops (Humulus lupulus) and chemical derivatives thereof. Hop derivatives (e.g., alpha acids, beta acids) may be purified from hops or may be chemically synthesized.

By “infestation” is meant the colonization of a site, such as a hive, or the parasitization of an organism by a pest.

By “inhibit” is meant slow, stabilize, reduce, or otherwise ameliorate the growth, proliferation or survival of a pathogen infection or symptom thereof in a host organism or population.

By “miticide” is meant an agent that inhibits a biological function of a mite.

By “miticidal activity” is meant any activity that inhibits the growth, reproduction, or survival of a mite or other acarid.

By “preventing a mite infestation” is meant reducing the success that a mite infestation will be established in an Apis colony.

By “suitable form for delivery to a honey bee or hive” is meant in a form that facilitates distribution of an effective amount of the hops derivative in the hive.

By “treating a mite infestation” is meant reducing, stabilizing, or slowing the growth of a mite population in an Apis colony.

### BRIEF DESCRIPTION OF THE DRAWINGS

FIG. 1 is a graph showing Varroa mite mortality and adult honey bee survival in response to hop product exposure.

FIG. 2 illustrates the number of 1^{st} to 2^{nd} instar diamondback moth larvae per plant.

FIG. 3 illustrates the number of 3^{rd} to 4^{th} diamondback moth larvae per plant.

FIG. 4 illustrates the number of total number of diamondback moth larvae per plant.

FIG. 5 illustrates the number of 1^{st} to 2^{nd} instar cabbage loopers per plant.

FIG. 6 illustrates the number of 3^{rd} to 5^{th} instar cabbage loopers per plant.

FIG. 7 illustrates the total number cabbage loopers per plant.

### DETAILED DESCRIPTION OF THE INVENTION

The present invention is directed to methods and compositions to prevent or treat a honey bee pathogen infection or hive infestation with a destructive acarid or insect pest. The invention is based, at least in part, on the discovery that naturally occurring components of hops are useful for inhibiting the growth of bacterial pathogens of honey bees. In addition, the invention provides methods for controlling Varroa mites that can weaken a hive or act as vectors for bacterial diseases. In further embodiments, the invention provides for the treatment or prevention of hive infestations with destructive insects, including Lepidopteran pests, such as the wax moth (Galleria mellonella),

### Apis

Honey bees are insects that pass through four life stages: the egg, larva, pupa and adult. Adult bees belong to one of three castes: queen, worker, or drone. The queen bee is the only female in the colony that is capable of reproduction and is responsible for all egg production. The worker bees are non-reproductive females who gather honey and care for the queen's progeny, or “brood.” The drones are male bees that mate with the queen. The life cycle, from egg to adult bee, takes twenty-one days for worker bees and twenty-four days for drones. The queen bee lays each egg in a single cell of the comb. The egg generally hatches into a larva on the fourth day, which continues its development within the cell. On the ninth day the cell with the developing larva is capped with wax and the larva undergoes pupal metamorphosis. On day twenty-one, a new adult worker bee emerges.

### Honey Bee Pathogens and Pests

American Foulbrood

The spore-forming, Gram-positive bacterium Paenibacillus larvae causes American foulbrood, a serious and fatal bacterial disease of honey bee larvae. American foulbrood is highly contagious and infects bee colonies throughout the world. Because control of the pathogen often requires destruction of infected colonies, American foulbrood causes considerable economic loss to beekeepers worldwide. Larvae in the first and second instars are highly susceptible to this disease, with the ingestion of as few as 10 infectious spores from virulent strains being sufficient to cause mortality (Brodsgaard et al., (1998) Apidologie 29: 569-578). American foulbrood is transmitted by spore-containing honey being fed to newly hatched larvae. The spores germinate in the midgut lumen. The dying larvae form creamy or dark brown, glue-like larval remains that provide the most obvious clinical symptom of American foulbrood. Infected individuals turn brown and then black, and the resultant mass becomes a hard scale of material deposited on the sides of cells within the hive.

Methods of diagnosing American foulbrood include the analysis of clinical symptoms within the hive including larval remains or scales, PCR methods (Alippi et al., Lett Appl Microbiol. 2004; 39(1):25-33; Dobbelaere et al., (2001) Apidologie 32:363-370; Govan et al., (1999) Appl. Environ. Microbiol. 65:2243-2245), DNA fingerprint analysis (Alippi et al., Lett Appl Microbiol. 2004; 39(1):25-33), as well as biochemical profiling (Neuendorf Microbiology 150 (2004), 2381-2390), bacteriophage sensitivity, immunotechniques and microscopy of suspect bacterial strains. Such methods are known in the art and are described, for example, by de Graaf et al., “Diagnosis of American foulbrood in honey bees: a synthesis and proposed analytical protocols,” Lett Appl Microbiol. 2006 December; 43(6):583-90. Infected bees generally die from the disease late in larval development. Bees mitigate American foulbrood through hygienic behaviour by adult workers and through larval resistance traits.

European Foulbrood

Melissococcus pluton, which is also referred to as by Melissococcus plutonis, is the causative agent of European foulbrood. European foulbrood is a bacterial infection of honeybee larvae that kills them when they are 4 or 5 days old. European foulbrood is caused by Melissococcus pluton (Bailey et al., J Appl Bacteriol. 1983; 55:65-69), which is a gram-positive bacterium formerly known as Bacillus pluton. Methods of diagnosing European foulbrood are known in the art and are described, for example, by Govan et al., “A PCR Detection Method for Rapid Identification of Melissococcus pluton in Honeybee Larvae,” Appl Environ Microbiol. 1998 May; 64(5): 1983-1985. In one embodiment, negative staining with nigrosin assists in the identification of bacteria resembling M. pluton. Colonies can be evaluated microscopically by identifying non-spore-forming, lanceolate coccus, occurring singly, in pairs, and in chains that typically measure 0.5 to 0.7 by 1.0 μm (Alippi et al., J Apic Res. 1991; 30:75-80; Shimanuki et al., “Diseases and pests of honey bees,” In: Graham J., editor; The hive and the honey bee. Hamilton, III: Dadant and Sons; 1992. pp. 1083-1151.). M. pluton may also be distinguished by scanning electron microscopy (Alippi et al., J Apic Res. 1991; 30:75-80), by enzyme-linked immunosorbent assays (Pinnock and Featherstone, J Apic Res. 1984; 23:168-170), with polyclonal antisera by using type strains (Allen and Ball, J Apic Res. 1993; 32:80-88), or using PCR or DNA fingerprinting (Govan et al., Appl Environ Microbiol. 1998 May; 64(5): 1983-1985).

Table 1 summarizes methods for distinguishing between European and American foulbrood infections.

TABLE 1

| Characteristics | American foulbrood | European foulbrood |
|---|---|---|
| Comb | Sealed brood, dark, | Unsealed brood, |
| | sunken or punctured | discolored, possibly |
| | cappings | punctured cappings |
| Age of Affected Brood | Older sealed larvae, | Younger larvae still |
| | pupae | in C shape |
| Color of Affected | Dull white, light | Dull white, light |
| Brood | brown to black | brown to black |
| Consistency of | Sticky to ropy in | Not sticky to ropy |
| Affected Brood | appearance | |
| Odor of Affected | “Glue pot” odor | Sour odor |
| Brood | | |
| Scale | Flat outstretched on | Twisted in cell as it |
| | side of cell; adheres | attacks younger |
| | tightly; may contain | larvae; does not |
| | adult characters | adhere |
| | (tongue, legs) | |

Chalkbrood

Ascosphaera apis is the fungal cause of the honey bee larval disease chalkbrood (Williams, D. L. (2000) A veterinary approach to the European honey bee (Apis mellifera). Vet J 160: 61-67; Hornitzky, M. (2001) Literature review of chalkbrood—a fungal disease of honeybees. Rural Industries Research and Development Corporation. RIRDC Publication 01/150; Kingston ACT, Australia). Spores of this fungus germinate within the digestive tract of bees, then begin fungal filamentous (mycelial) growth during the last instar of larval development. Dead larval and pupal bees appear chalky due to growth throughout the bee of mycelia. These chalky ‘mummies’ are highly infectious, and spores of this fungus often reinfect colonies via stored food supplies or direct transport to younger larvae by adult bees working within the nest. Adult bees reduce the effects of the fungus on the colony by identifying and removing diseased individuals.

Chalkbrood, American and European foulbrood may occur alone or in combination with a Varroa mite or wax moth infestation. Pest infestations weaken the bee colony and render it more susceptible to pathogens. In addition, the exoskeletal wounds produced by Varroa mites provide an entry for bacterial or fungal infections. Compositions of the invention are useful not only for treating honey bee bacterial infections, but also for controlling mite or Lepidopteran infestations.

### Acarids

Parasitic mites that prey on honey bees include Varroa mites (e.g., Varroa destructor, Varroa jacobsoni) and tracheal mites (e.g., Acarapis woodi). Varroa mites are ectoparasites that feed on bee hemolymph, and infest wild and domestic honey bee colonies. Varroa mite reproduction begins when the adult female mite enters a brood cell shortly before it is capped. Drone brood, which is reared in larger cells than worker brood, is preferentially targeted for mite infestation. The female mite feeds on the larval hemolymph prior to depositing her eggs. The Varroa eggs eclose under the sealed cell, and the developing mites feed on the bee pupa. The first egg laid by the female Varroa develops into a male. Subsequent eggs develop into females that mate with their brother. The mated female mites along with their mother are released from the capped cell when the bee emerges. The female mites typically attach to adult bees between the abdominal segments or between body regions, where they feed on the bees' hemolymph. Adult bees serve as intermediate hosts and as a means of transport to new sites of infestation.

Desirably, the invention provides miticides that control Varroa mite infestations and are also useful for preventing bacterial infections that occur in combination with such infestations, and controlling the spread of disease.

### Wax Moth Infestations

The greater wax moth (Galleria mellonella) is one of the most serious and destructive insect pests of unprotected honey bee combs. Wax moths infest stored equipment and will invade colonies whose worker bee population have been weakened by disease, queenlessness, failing queens, pesticide kills or starvation. Female moths lay 300 to 600 eggs in clusters on honeybee combs or in small cracks in hive material. The eggs hatch within 3-5 days and the wax moth larvae burrow into the comb feeding on the wax, larval skins, pollen and honey. As the wax moth larvae chew through the comb they spin a silk lined tunnel through the cell walls and over the face of the comb. These silk threads can trap emerging bees in their cells where they die of starvation. This phenomenon is termed galleriasis. In severe infestations, the wax comb, wooden frames, and sides of the hive bodies can be heavily damaged.

As reported herein, hops derivatives are effective at controlling a variety of pests, including Lepidoptera larvae, and are expected to be effective against wax moth larvae, as well. Compositions of the invention can be used for controlling a Lepidopteran infestation alone, or in combination with a Varroa mite infestation or a European or American foulbrood infection.

### Small Hive Beetles

The small hive beetle (Aetina tumida), which is native to South Africa, was discovered in Florida in 1998. The larva of the small hive beetle is the life stage that is most damaging to the honey bee hive. The beetle can multiply to huge numbers within infested colonies where it eats brood, destroys combs and if uncontrolled ultimately destroys the colony. In fact, over 10,000 colonies were destroyed in the year following the beetles identification in the U.S. Both larvae and adults eat bee eggs and brood, but they will also eat pollen and honey. As the larvae grow they burrow through brood combs causing great damage and ultimately consuming the colony's brood nest. In heavy infestations, tens of thousands of small hive beetle larvae may be present within the colony with as many as 30 larvae present in a single cell. Defecation of adult beetles and larvae in honeycomb causes the honey to ferment and drip out of cells. Affected combs become slimy and have a characteristic odor reminiscent of “rotten oranges”. Small hive beetle damage renders the honey unfit for sale. In addition, the ruined combs are repellent to bees and can cause the bees to abandon the hive. Hives already infested with Varroa mites or bacterial disease may be particularly vulnerable to the increased stress associated hive beetle infestation.

### Treatment and Prevention of Pathogen Infections

The invention provides compositions containing hop derivatives, including alpha and/or beta acids that are used to prevent or reduce a pathogen infection (e.g., bacterial or fungal infection) or hive infestation (e.g., wax moth, Varroa mite). Compositions of the invention have biocidal activity and are therefore useful to reduce, stabilize, slow, or inhibit the spread of a pathogen infection or pest infestation in larvae, bees, and hives. Methods for identifying and/or characterizing a pathogen infection are known in the art and are described herein. In particular, methods for identifying a pathogen infection include methods that involve identifying or characterizing the genotype of a pathogen by PCR amplification, DNA fingerprinting, nucleic acid sequencing, or hybridization with polynucleotide probes. Other methods for identifying a pathogen include the biochemical or clinical characterization of the pathogen. Biochemical characterization methods include immunoassays, bacterial staining, and the like. Clinical characterization of a pathogen infection includes microscopic examination of bacteria or bacterial colony morphology. Alternatively, the clinical symptoms of infection within the hive may be evaluated by an inspection of the hive that includes characterizing the appearance of dead or dying larvae or the smell of an infected hive.

Compositions of the invention reduce the level of pathogen infection within a bee population or hive. A number of parameters are indicative of the level of pathogen infection present in a bee colony including, but not limited to, larval death, the number of pathogens (e.g., bacteria) present in dead or infected larvae; the number of bees reared in a hive relative to the number reared in an uninfected hive; or a reduction in the size of bees reared in a hive relative to the size of bees reared in a hive without an infection. Thus, bee size or weight can be used as one measure of pathogen infection. Alternatively, the presence or number of bacterial spores in honey produced by the hive may serve as a measure of the level of pathogen infection. The amount of honey produced in an infected hive is typically less than that produced in a healthy hive; accordingly, honey production serves as yet another measure of the level of pathogen infection; and finally, severe infections result in complete loss of colonies. Thus, loss of colonies can be a measure of the level of pathogen infection in the hive.

In one embodiment, a biocide of the invention prevents or treats a bacterial or fungal infection in a larvae, adult bee, or hive by reducing larval death by at least 10%, 25%, 50%, 75% or even by 100%. In another embodiment, a biocide of the invention increases the survival of larvae in a hive by at least 25%, 50%, 60%, or 70%, or even by as much as 80%, 90% or 100%. Preferably, a biocide of the invention reduces the growth, proliferation, or survival of a honey bee bacterial or fungal pathogen (e.g., Melissococcus plutonis, Paennibacillus larvae, Ascosphaera apis) by at least 10%, 25%, 50%, 75%, 80%, 90%, or even 95% or 100% in an standard assay used to measure bacterial or fungal growth in culture (e.g., an agar diffusion assay).

An agar diffusion assay is one indicator of biocidal activity. In brief, a disc of filter paper containing a composition of the invention is used to contact a bacterial or fungal growth medium and the growth of bacteria (e.g., Melissococcus plutonis, Paennibacillus larvae, Ascosphaera apis) is measured to determine a zone of inhibition surrounding or under the filter paper. Methods for culturing such Melissococcus plutonis, Paennibacillus larvae, and Ascosphaera apis are known in the art. See, for example, Hornitzky et al., “Procedures for the culture of Melissococcus pluton from diseased brood and bulk honey samples,” Journal of Apicultural Research (1998) 37 (4):293-294; and Bailey (1982). Reclassification of Streptococcus pluton (White) in a new genus Melissococcus, as Melissococcus pluton, J. Appl. Bacteriol., 53, 215-217.

In brief, Melissococcus pluton is cultivated on a medium comprising: yeast extract or certain peptones; cysteine; glucose or fructose; soluble starch; KH₂PO₄ at pH 6.6; and agar under anaerobic conditions at 30° C. Methods of culturing Paenibacillus larvae involve culturing the bacteria on Columbia blood agar at 37° C. as described by Bakonyi Appl Environ Microbiol. 2003 March; 69(3):1504-10; and by Steinkraus, K. H., and R. A. Morse. 1996 Acta Biotechnol. 16:57-64. Methods of culturing Ascosphaera apis are described by Liu et al., Biomedical and Life Sciences 116:23-28, 1991. In brief, the fungus is cultured on YGPSA plates for 7 days at 30° C. in darkness. YGPSA contains in g/l: yeast extract 10 g, glucose 10 g, KH₂PO₄ 13.5 g, soluble starch 10 g, and agar 20 g; pH 6.6. Culture plates are streaked with dilute aqueous suspensions of bacteria or fungus, diseased larvae, or diseased larval mid-guts.

Screening methods are used to identify concentrations of hop derivatives that will be bactericidal. In one embodiment, the agar diffusion is used to determine the minimum inhibitory concentration (MIC) of alpha acids or beta acids that inhibit bacterial growth. Preferably, the MIC is at least about 500 ppm, 1000 ppm, 1500 ppm, or 2000 ppm. Even more preferably, a dose is selected that has bactericidal activity while minimizing any lethal effect on adult bees.

In other embodiments, a composition of the invention reduces the growth, proliferation, or survival of a Lepidopteran or acarid pest in a hive.

### Biocidal Screening

Commercial products that are currently being used to control bacterial infections or hive infestations can have adverse effects on humans. For example, tetracycline or tetracycline break down products can contaminate honey intended for human consumption. Conventional antibiotics include terramycin, which is typically used to prevent American foulbrood infections. Terramycin® (oxytetracycline HCL) is the only drug approved for use as a preventive treatment against American foulbrood. Because no effective treatment is available for the permanent control of American foulbrood all infected honey and combs are burnt to prevent the disease from spreading to other colonies. Oxytetracycline hydrochloride is also used to prevent or treat European foulbrood infections. Commercial products include Oxymav 100® (active ingredient 100 g/kg), Tetravet 100® (100 g/kg), and Broodmix® (10 g/kg). The use of such antibiotics often results in honey contamination and the development of resistant strains of bacteria.

In contrast to conventional treatments or prophylactics, compositions of the invention contain safe natural products derived from hops. Hops have been used for centuries to flavor beer; thus, formulations comprising hop derivatives are generally safe. Biocidal compositions (e.g., compositions that inhibit the growth of bacterial or fungal pathogens or destructive insects) of the invention will not adversely affect human bee keepers or honey intended for human consumption.

Biocides of the invention contain concentrations of hop derivatives that have few or no adverse effects on honey bees during any of their life stages, but are effective in inhibiting bacterial or fungal growth, proliferation, or survival, as well as controlling a hive infestation (e.g., a wax moth or Varroa mite infestation). As reported herein, tetrahydroisoalpha acids, beta acids, and hexahydroisoalpha acids, which are hop derivatives, each delivered at 1000 ppm inhibited bacterial growth in an agar diffusion assay while causing 0% lethality in adult bees. In particular, beta acids inhibited the growth of Melissococcus plutonis and Paennibacillus larvae; tetrahydroisoalpha acids and hexahydroisoalpha acids inhibited the growth of Paennibacillus larvae. In one approach, bee pathogens are exposed to varying concentrations of hop derivatives to identify those concentrations that inhibit bacterial or fungal growth. Adult honey bees are then exposed to concentrations of hop derivatives having bactericidal or fungicidal activity to identify those that have a minimal effect on honey bee survival. In one approach, bacteria, fungus, or destructive insects, such as wax moths, are exposed to varying concentrations of hop derivatives to identify those concentrations that have biocidal activity, i.e., those that kill 50% to 100% of the exposed pathogen or destructive insects. Adult honey bees are then exposed to concentrations of hop derivatives having biocidal activity to identify those that have a minimal effect on honey bee survival. Preferably, at least 75%, 80%, 85%, 90%, 95%, or 100% of adult bees will survive following exposure to a biocidal composition. In a similar approach, the effect of hop derivatives on honey bee reproduction is assessed. Screening assays are used to determine the concentration of a biocide that has only a minimal effect on honey bee reproduction.

### Hop Derivatives

A hop derivative is a compound that occurs naturally in a hop plant (Humulus lupulus) or is chemically derived (either through natural biosynthetic processes (e.g., living organism metabolism (e.g., mammal, plant, bacteria)) or by synthetic processes using human intervention (e.g., chemical synthesis). Compositions of the invention include one or more compounds derived from hops. Of particular interest are the hop acids. Hops contain two major organic acid classes, alpha acids and beta acids. Hop acids are the bitter acid components of hops that are used in beer making. There are three major analogs for alpha acids, humulone, cohumulone, and adhumulone, and three major analogs for beta acids, lupulone, colupulone, and adlupulone. The percentages of the analogs present in the alpha acids and beta acids are variety-dependent. Thus, hop derivatives and hop products typically contain one or a mixture of these analogs. The percentage of analog present is dependent on the hop variety used to produce the derivative or product. Alpha acids and beta acids can be prepared by purification from natural hops and also by chemical synthesis according to traditional methods. Exemplary hop derivatives include beta acids, hexahydrobeta acids, rhoisoalpha acids, isoalpha acids, tetrahydroisoalpha acids, hexahydroisoalpha acids, magnesium salts of rhoisoalpha acids and magnesium salts of beta acids. Compositions comprising hop derivatives are also available commercially. John I. Haas, Inc. products containing hop derivatives include BetaStab® 10A, also referred to hereafter as Betacide, Redihop®, Isohop®, Tetrahop Gold®, Hexahop Gold®, magnesium salts of rhoisoalpha acids, and magnesium salts of beta acids. The active ingredients in these products are beta acids, rhoisoalpha acids (RIAA), isoalpha acids (IAA), tetrahydroisoalpha acids (THIAA), hexahydroisoalpha acids (HHIAA), magnesium salts of rhoisoalpha acids (MgRIAA) and magnesium salts of beta acids, respectively. For convenience, the identities of these products are also listed in Table 2. These products and/or hop derivatives are typically diluted to a desired concentration for use in the methods of the invention.

Plant extracts are often used for the purification of compounds from plants (e.g., hops). An extract can be prepared by drying and subsequently cutting or grinding the dried material. The term “extract” refers to a concentrated preparation of the essential constituents of a plant, such as hops. Typically, an extract is prepared by drying and powderizing the plant. Optionally, the plant, the dried plant or the powderized plant may be boiled in solution. The extract may be used in liquid form, or it may be mixed with other liquid or solid herbal extracts. Alternatively, the extract may be obtained by further precipitating solid extracts from the liquid form. The extraction process may then be performed with the help of an appropriate choice of solvent, typically ethanol/water mixture, methanol, butanol, iso-butanol, acetone, hexane, petroleum ether or other organic solvents by means of maceration, percolation, repercolation, counter-current extraction, turbo-extraction, or by carbon-dioxide supercritical (temperature/pressure) extraction. The extract may then be further evaporated and thus concentrated to yield by means of air drying, spray drying, vacuum oven drying, fluid-bed drying or freeze-drying, the extract product.

Crude extracts are tested for bactericidal activity as described herein. Further fractionation of a positive lead extract having miticidal activity is necessary to isolate chemical constituents responsible for the observed effect. Thus, the goal of the extraction, fractionation, and purification process is the careful characterization and identification of a chemical entity within the crude extract that disrupts a mite biological function. Methods of fractionation and purification of such heterogeneous extracts are known in the art. If desired, compounds shown to be useful as bactericidals are chemically modified according to methods known in the art.

Numerous methods are available for the chemical synthesis of candidate compounds. Such compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); L. Paquette, ed., Encyclopedia of Reagents for Organic Synthesis, John Wiley and Sons (1995); and M. Verzele and D. De Keukeleire, Chemistry and Analysis of Hop and Beer Bitter Acids, Elsevier: Amsterdam (1991). Chemically synthesized alpha and beta acids can be separated from a reaction mixture and further purified by a method such as column chromatography, high pressure liquid chromatography, or recrystallization. As can be appreciated by the skilled artisan, further methods of synthesizing the compounds herein will be evident to those of ordinary skill in the art. Additionally, the various synthetic steps may be performed in an alternate sequence or order to give the desired compounds.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein. All such isomeric forms of such compounds are expressly included in the present invention. All crystal forms of the compounds described herein are expressly included in the present invention. As used herein, the compounds of this invention, including the compounds of formulae described herein, are defined to include derivatives. Derivatives include compounds of the invention that are modified by appending appropriate functionalities to enhance desired properties.

Acceptable salts of the compounds of this invention include those derived from acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic acid, may be employed in the preparation of salts useful as intermediates in obtaining the compounds of the invention and their acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

Lower or higher doses than those recited herein may be required to effectively inhibit bacterial growth without adversely affecting honey bees. Specific dosage and treatment regimens are determined empirically as described herein. Compositions of the invention are also useful for preventing the establishment of an pathogen infection, for treating an established pathogen infection, and for maintaining the health of a hive previously treated for an pathogen infection.

### Formulations

Hop derivatives can be provided to bees or bee hives in a number of convenient formulations. In general, strategies for dispersing a therapeutic or prophylactic agent within the hive rely on: i) providing the agent in a food source (e.g., a liquid or solid food); ii) providing the agent in a composition that will induce hygienic behavior designed to remove the composition from the colony (a packet designed to be torn apart by the bees); or iii) providing the agent in a form that the bees will inadvertently distribute throughout the colony (e.g., a tracking powder provided at an entrance to the hive). Formulations of the invention are used to target bacteria or fungi, or bacterial or fungal spores on the body of adult bees, in the brood cell, in the honey, or in the hive. Desirably, the composition of the invention is active in the hive for at least 1 week, 2 weeks, 3 weeks, 4 weeks, or for 1, 2, or 3 months. Where treatment or prevention of a mite or wax moth infestation is desired, compositions of the invention are desirably present throughout the life cycle of the mite or moth. Where activity is maintained for a shorter period (e.g., seven, fourteen, twenty-one, or thirty days), repeated administration of a composition of the invention may be required. Compositions that are active for longer periods (e.g., two, three, six, nine, or twelve months) are also envisioned. Such compositions may be used for the long-term treatment or prevention of a bacterial or fungal infection or mite or wax moth infestations.

Powdered Formulations

Conventional treatments for mite infestations are introduced into the beehive on plastic non-biodegradable strips that are about 1″ wide, 9″ long and ¼″ thick. Similar means could be used for the delivery of hop derivatives. In one embodiment, a composition comprising a hop derivative is provided in a powdered formulation as a strip or a patty. A substrate material is coated with a powdered formulation of hop acids, and the coating is subsequently encased in a layer of a substance that is attractive to bees, such as powdered sugar. This strip is placed inside the beehive where the adult bees chew into the powdered sugar and expose the powdered hop acids. In another approach, the hop acids are mixed with powdered sugar and the mixture is formed into a patty. In another approach, the powdered mixture is delivered to the hive within a semi-permeable pouch that resembles a “teabag”. To rid the hive of this foreign object, the bees rip up the pouch, thereby releasing the powder. The powdered hop acids get onto the body of the adult bees and are distributed throughout the hive, thereby contacting bacteria, bacterial spores, or mites present on the adult bees and inhibiting the bacterial infection and/or mite infestation. Alternatively, the hop acids are consumed by the larvae and thereby treat the bacterial infection and/or mite infestation.

Encapsulated Formulations

In one approach, a hop derivative is provided in an encapsulated formulation (liquid or powder). Preferably, a hop derivative in liquid or powder form is encapsulated in a coating that breaks down slowly inside the beehive. The coating provides for the long-term release of the hop derivative. Preferably, the composition is released over the course of two to six weeks (e.g., two, three, four, five, six weeks). Specific materials suitable for use in capsule materials include, but are not limited to, porous particulates or substrates such as silica, perlite, talc, clay, pyrophyllite, diatomaceous earth, gelatin and gels, polymers (e.g., polyurea, polyurethane, polyamide, polyester, etc.), polymeric particles, or cellulose. These include, for example, hollow fibers, hollow tubes or tubing which release a hop derivative or other compound specified above through the walls, capillary tubing which releases the compound out of an opening in the tubing, polymeric blocks of different shapes, e.g., strips, blocks, tablets, discs, which release the compound out of the polymer matrix, membrane systems which hold the compound within an impermeable container and release it through a measured permeable membrane, and combinations of the foregoing. Examples of such dispensing compositions are polymer laminates, polyvinyl chloride pellets, and microcapillaries. Encapsulation methods suitable for use in apiculture are described, for example, by Rieth et al., Journal of Apiculture Research 25(2):78-84 (1986).

Encapsulation processes are typically classified as chemical or mechanical. Examples of chemical processes for encapsulation include, but are not limited to, complex coacervation, polymer-polymer incompatibility, interfacial polymerization in liquid media, in situ polymerization, in-liquid drying, thermal and ionic gelation in liquid media, desolvation in liquid media, starch-based chemistry processes, trapping in cyclodextrins, and formation of liposomes. Examples of mechanical processes for encapsulation include, but are not limited to, spray drying, spray chilling, fluidized bed, electrostatic deposition, centrifugal extrusion, spinning disk or rotational suspension separation, annular-jet encapsulation, polymerization at liquid-gas or solid-gas interface, solvent evaporation, pressure extrusion or spraying into solvent extraction bath.

Microcapsules are also suitable for the long-term release of biocides. Microcapsules are small particles that contain a core material or active ingredient surrounded by a coating or shell. The size of the microcapsule typically varies from 1 to 1000 microns with capsules smaller than 1 micron classified as nanocapsules and capsules larger than 1000 microns as macrocapsules. Core payload usually varies from 0.1 to 98 weight percent. Microcapsules can have a variety of structures (continuous core/shell, multinuclear, or monolithic) and have irregular or geometric shapes.

In another approach, the hop derivative is provided in an oil-based delivery system. The oil-hop derivative mix is deposited on a solid substrate and the substrate containing the hop derivative is placed into the hive where it subsequently contacts and kills the bacteria. Oil release substrates include vegetable and/or mineral oils. In one embodiment, the substrate also contains a surface active agent that renders the composition readily dispersable in water; such agents include wetting agents, emulsifying agents, dispersing agents, and the like.

Biocides of the invention can also be provided as emulsions. Emulsion formulations can be found as water in oil (w/o) or oil in water (o/w). Droplet size can vary from the nanometer scale (colloidal dispersion) to several hundred microns. A variety of surfactants and thickeners are usually incorporated in the formulation to modify the size of the droplets, stabilize the emulsion, and modify the release.

Alternatively, biocides of the invention may also be formulated in a solid tablet and comprise (and preferably consist essentially of) an oil, a protein/carbohydrate material (preferably vegetable based), a sweetener and an active ingredient useful in the prevention or treatment of a bacterial infection in a honey bee. Methods for making such compositions are known in the art and are described, for example, in U.S. Patent Publication No. 20060008492. In one embodiment the invention provides a solid tablet and comprise (and preferably consist essentially of) an oil, a protein/carbohydrate material (preferably vegetable based), a sweetener and an active ingredient (e.g., hops α and/or β acid) useful in the prevention or treatment of diseases in insects. Tablets typically contain about 4-40% (e.g., 5%, 10%, 20%, 30%, 40%) by weight of an oil (e.g., plant oil, such as corn, sunflower, peanut, olive, grape seed, tung, turnip, soybean, cotton seed, walnut, palm, castor, earth almond, hazelnut, avocado, sesame, croton tiglium, cacao, linseed, rape-seed, and canola oils and their hydrogenated derivatives; petroleum derived oils (e.g., paraffins and petroleum jelly), and other water immiscible hydrocarbons (e.g., paraffins). The tablets further contain from about 5-40% (e.g., 5%, 10%, 20%, 30%, 40%) by weight of a vegetable-based protein/carbohydrate material. The material contains both a carbohydrate portion (e.g., derived from cereal grains such as wheat, rye, barley, oat, corn, rice, millet, sorghum, birdseed, buckwheat, alfalfa, and mielga, corn meal, soybean meal, grain flour, wheat middlings, wheat bran, corn gluten meal, algae meal, dried yeast, beans, rice) and a protein portion. While the relative fraction of each portion making up the material may vary, the material should include at least a portion of carbohydrate and protein.

The tablets also contain between about 10-75% (10, 15, 20, 25, 50, 75%) by weight of a sweetener. As used herein, the term “sweetener” generally refers to both natural and artificial sweeteners. Preferably, the sweetener is a sugar such as glucose, fructose, sucrose, galactose, lactose, and reversed sugar. The sugar is preferably selected from the group consisting of granulated sugar (white sugar), brown sugar, confectioner's sugar, impalpable sugar, icing sugar, and combinations thereof. Alcohols such as glycerin and complex carbohydrates, such as starches may also be used as the “sweetener” ingredient. The sweetener is used primarily as an attractant for the insects, however the sweetener also helps to impart a granular structure to the tablets, especially when the sweetener is a sugar. As previously discussed, this granular structure permits the tablet to crumble over time upon the exertion of sufficient forces.

Optionally, various excipients and binders can be used in order to assist with delivery of the active ingredient or to provide the appropriate structure to the tablet. Preferred excipients and binders include anhydrous lactose, microcrystalline cellulose, corn starch, magnesium estearate, calcium estearate, zinc estearate, sodic carboxymethylcellulose, ethyl cellulose, hydroxypropyl methyl cellulose, and mixtures thereof.

Tablets according to the present invention are manufactured by mixing all of the ingredients together and then compressing the mixture into a tablet of desired shape and size for a particular application. Preferably, the tablet is discoid in shape with a diameter of between about 2-5 inches and a thickness of from about 0.5-2 inches. The pressing may be accomplished by a manual or automatic pressing device. The pressure exerted on the mixture should be sufficient so as to form the tablet into a self-sustaining body.

Methods of delivering an active ingredient to an insect according to the present invention comprise the steps of providing a solid tablet containing the active ingredient as previously described and placing the tablet in a location where the insect may come into direct contact therewith. Preferably, the insects to which the active is being delivered are honeybees. In treating honeybees that are generally colonized in a manufactured bee hive, the tablet is preferably placed inside the hive.

Over the next several weeks after the tablet is placed into the hive, the bees chew and crumble the tablet exposing the active ingredient to the other bees. The crumbs fall through the brood box away from the honey supers. Preferably, the entire tablet is disintegrated in about 30-45 days.

Biocides of the invention can also be delivered in the form of syrups that are attractive to bees and induce feeding behavior. The syrups for use in the invention preferably comprise sugar and water. Particularly preferred are 50% w/v sucrose solutions. A liquid composition is formed by dispersing hops acids in a sugar syrup comprising 50% sucrose in water. The composition is used as a feed supplement for the bees and can be placed at a suitable location in or near a hive.

Biocides of the invention can also be delivered in packets suitable for inducing hygienic behavior in bees. Such packets are prepared by enclosing a fine powder of hops acids and sugar in a porous material capable of being torn apart by bees. Preferably, the porous material is made of waxed paper or filter paper. Suitable filter papers include those comprising abaca fibers, wood pulp and cellulose rayon fibers. If desired, the paper is coated with polyethylene mixed with copolymers, polypropylene mixed with copolymers or 100% polypropylene.

In other embodiments, biocides are prepared in a dusting composition. Dusting compositions are typically prepared by grinding sugar to a fine powder and mixing into the powder hops acids. The dusting can be applied directly to the combs within the hive, or to the interior surfaces of the hive, or may be applied directly to a bee cluster.

Alternatively, the biocides are prepared in a liquid spray composition that is formed by dispersing hops acids in any suitable liquid. Preferably, the hops acids are dispersed in water. If desired, the spray composition also includes a surfactant that allows the spray to be dispersed efficiently without clogging the spraying apparatus. The composition can be used to spray the hive interior, or the comb, or can be used to spray bee clusters directly.

In another approach, biocides of the invention are delivered in the form of a vapor. Methods for delivering such vapors to a hive are described, for example, in U.S. Patent Publication No. 20020151249.

### Biocide Delivery

Devices for delivering biocidal agents to bees or to a bee hive are known in the art. Such delivery devices include strips, controlled release strips, tablets, reservoirs, polymer discs, trays, sprayers, and evaporation devices. If desired, the delivery device is provided in a biodegradable form. In particular, devices suitable for delivering a composition of the invention to a bacteria, parasitic mite, to a honey bee, or to a honey bee hive are described, for example, in U.S. Patent Publication Nos. 20040229542, 20050090560, and 20020151249. Dispensing means and suitable compositions for controlled release are described in U.S. Pat. Nos. 6,843,985; 6,037,374; 5,750,129; 4,775,534; 5,849,317; 5,348,511; 6,037,374; 3,577,515, which are incorporated herein by reference in their entirety.

### Kits

The invention provides kits for the treatment or prevention of bacterial infections and pest infestations in a honey bee or hive. In other embodiments, the invention is used to treat European or American foulbrood or chalkbrood infections that occur together with a Varroa mite infestation. In one embodiment, the kit includes a composition containing an effective amount of a hop derivative in a form suitable for delivery (e.g., those delineated herein) to a site of bacterial infection or mite infestation (e.g., bee hive). In some embodiments, the kit comprises a container which contains a biocide (e.g., a miticide, bactericide, insecticide); such containers can be boxes, ampoules, bottles, vials, tubes, bags, pouches, blister-packs, or other suitable container forms known in the art. Such containers can be made of plastic, glass, laminated paper, metal foil, or other materials suitable for holding miticides.

If desired the bactericide of the invention is provided together with instructions for administering it to a site of bacterial infection. In other embodiments, the instructions will include methods for delivering it to a hive to control an acarid or Lepidopteran infestation. The instructions will generally include information about the use of the composition for the treatment or prevention of an bacterial infections. In other embodiments, the instructions include at least one of the following: description of the biocide; dosage schedule and administration for treatment or prevention of a bacterial infections; precautions; warnings; description of research studies; and/or references. The instructions may be printed directly on the container (when present), or as a label applied to the container, or as a separate sheet, pamphlet, card, or folder supplied in or with the container.

### EXAMPLES

### Example 1

### M. plutonis Assay with Hop Beta and Alpha Acids

The disk-diffusion method (Kirby-Bauer) is suitable for testing bacterial or fungal isolates for susceptibility to a biocide comprising a hop derivative. In brief, an agar plate suitable for bacterial or fungal growth is uniformly inoculated with a test organism and a paper impregnated with a fixed concentration of a hop derivative is placed on the agar surface. Growth of the organism and diffusion of the antibiotic commence simultaneously resulting in a zone of inhibition in which the amount of biocide exceeds inhibitory concentrations. The diameter of the inhibition zone is a function of the amount of drug in the disk and susceptibility of the microorganism.

A suspension of Melissococcus plutonis was grown up in MPM media.

| | | |
|---|---|---|
| Glucose | 10.0 | g |
| Soluble starch | 2.0 | g |
| Peptone (Oxoid L37) | 2.5 | g |
| Yeast Extract (Oxoid L21) | 2.5 | g |
| Neopeptone, DIFCO (BD 211681) | 5.0 | g |
| Trypticase Peptone (BD 211921) | 2.0 | g |
| 1 M Phosphate buffer, pH 6.7 | 50.0 | ml |
| L-Cysteine•HCl | 0.25 | g |
| Distilled water | 950.0 | ml |

The medium was adjusted to a final pH 7.2. Medium was dispensed into tubes flushed with 90% N₂, 10% CO₂ and immediately plugged with butyl rubber stoppers, then autoclaved at 121° C. for 15 minutes.

A tube of MPM media was inoculated with M. plutonis and cultured for several days under anaerobic conditions at 30° C. until the culture medium appeared turbid. The medium was then subcultured and spread on Trypticase Soy Agar (TSA) agar plates with 5% sheep blood and reincubated anaerobically at 30° C. until growth appeared. A 25×25 mm square sterile filter paper was dipped in Tetrahop Gold® formulation (9% tetrahydroisoalpha acids), Hexahop 95® formulation (5% hexahydroisoalpha acids and 5% tetrahydroisoalpha acids) or Betastab 10A® formulation (10% beta acids), which were diluted as follows:

BetaStab 10A® 1 ml into 99 ml H₂0;

Tetrahop Gold® 1.1 ml into 98.9 ml of H₂0;

Hexahop 95 0.5 ml into 99.5 ml sterile water.

The plate was incubated for 3 days in presence of filter paper and the zone of growth inhibition was measured. 1000 ppm of beta acids inhibited M. plutonis. The zone of inhibition for beta acids was 6 mm. 1000 ppm of hexahydroisoalpha acids and tetrahydroisoalpha acids inhibited M. plutonis growth directly under the disc.

### Example 2

### Paenibacillus larvae Assay with Hop Beta and Alpha Acids

A suspension of Paenibacillus larvae was grown up in Brain Heart Infusion (BHI) broth at 35-37° C. grown under aerobic conditions overnight until turbid then subcultured to BHI agar. The culture plate was incubated for 3 days in the presence of filter paper. The bacterial suspension was streaked over the surface of the BHI agar to obtain uniform growth and the plates were dried. Filter paper discs were treated with Tetrahop Gold® formulation (9% tetrahydroisoalpha acids), Hexahop 95® formulation (5% hexahydroisoalpha acids and 5% tetrahydroisoalpha acids) or Betastab 10A® formulation (10% beta acids), which were diluted as follows:

BetaStab 10A® 1 ml into 99 ml H₂0;

Tetrahop Gold® 1.1 ml into 98.9 ml of H₂0;

Hexahop 95 0.5 ml into 99.5 ml sterile water.

The plates were incubated overnight, and the diameter of the zone of growth inhibition around each disk was measured. The zone of inhibition for each of these formulations follows: beta acids was 6 mm; tetrahydroisoalpha acids was 2 mm; hexahydroisoalpha acids and tetrahydroisoalpha acids was 1 mm.

### Example 3

### Hop Beta and Alpha Acids Used in Miticide Screening

Beta acids, alpha acids, and a combination of beta and alpha acids were screened for efficacy as miticides. Liquid test products containing beta acids were provided in a BetaStab 10A® formulation (10% beta acids) hereinafter called “Betacide”. Liquid test products containing alpha acids were provided in a Redihop® formulation (30% rhoisoalpha acids), Isohop® formulation (30% isoalpha acids), Tetrahop Gold® formulation (9% tetrahydroisoalpha acids), Hexahop Gold® formulation (5% hexahydroisoalpha acids and 5% tetrahydroisoalpha acids). A combination of alpha and beta acids were prepared by mixing equal parts Redihop® and Betacide. Powdered test products containing beta acids were provided by a magnesium salt formulation of beta acids. Powdered test products containing alpha acids were provided by magnesium salt formulations of Redihop®, Tetrahop Gold® and Hexahop Gold®.

Tests were carried out using the concentrations of beta, alpha, or beta and alpha acid combinations indicated in Table 2.

Specifically, in Tests 1-4: 5% beta acids as Betacide test solution, 15% rhoisoalpha acids as Redihop® test solution, and a 2.5% beta acids/7.5% rhoisoalpha acids combination was used.

In Tests 5-8, 4% beta acids as Betacide test solution, 30% rhoisoalpha acids concentration as Redihop® test solution, and a 2% beta acids/15% rhoisoalpha acids combination were used.

In Tests 9-12, 4% beta acids as Betacide test solution, 30% rhoisoalpha acids concentration as Redihop® test solution, and a 2% beta acids/15% rhoisoalpha acids combination were used.

In Tests 13-15, 30% isoalpha acids as Isohop®, 9% tetrahydroisoalpha acids as Tetrahop Gold®, and a combination of 5% tetrahydroisoalpha acids and 5% hexahydroisoalpha acids from Hexahop Gold® were used.

In Tests 16-19, 4.3% and 8.5% beta acids as a magnesium salt, and 65.5% rhoisoalpha acids as a magnesium salt of Redihop® were used.

In Tests 20-22, 25.3% tetrahydroisoalpha acids as a magnesium salt of Tetrahop Gold®, and a combination of 12.2% each of tetrahydroisoalpha acids and hexahydroisoalpha acids from magnesium salts of Hexahop Gold® were used.

### Example 4

### Miticide Screening Assays

Tests using liquid hop products were conducted by absorbing one milliliter of test solution onto a filter paper in a Petri dish. Tests using the powdered hop products (magnesium salts) were conducted by spreading 0.5 gm of test powder evenly over filter paper in a Petri dish. Five to ten Varroa mites were then placed on the treated filter paper and mite survival was determined at one, four or five and twenty-four hours hour time points. Similar methods were used to evaluate the effect of the test compounds on adult honey bee survival. Adult honey bee survival was scored after twenty-two hours exposure to test product. Five to ten adult honey bees were placed in Petri dishes containing treated filter paper. Filter paper treated with water (for liquid test solutions) or cornstarch (for powdered test solutions) was used as a negative control for tests with the mites and the adult honey bees. All trials were replicated four times.

Table 2 outlines the tests and results of testing various hop products for miticidal activity.

TABLE 2

| Test | | Active Ingredient | Product Test acid % | | | | % Mortality/Exposure Time | | | |
|---|---|---|---|---|---|---|---|---|---|---|
| Number | Product | (ai) | Conc. % | Alpha | Beta | Diluent | Mites | Hours | Bees | Hours |
| 1 | deionized water | none | NA | NA | NA | none | 7 | 4 | 0 | 22 |
| 2 | Betacide | beta acids | 10 | NA | 5 | deionized water | 73 | 4 | 20 | 22 |
| 3 | Redihopâ | rhoisoalpha acids | 30 | 15 | NA | deionized water | 21 | 4 | 0 | 22 |
| 4 | RedihopÒ + Betacide as in test 1 + test 2 | | 30 + 10 | 7.5 | 2.5 | deionized water | 43 | 4 | 7 | 22 |
| 5 | deionized water | none | NA | NA | NA | none | 7 | 4 | 0 | 24 |
| 6 | Betacide | beta acids | 10 | NA | 4 | deionized water | 87 | 4 | 0 | 24 |
| 7 | Redihopâ | rhoisoalpha acids | 30 | 30 | NA | deionized water | 68 | 4 | 0 | 24 |
| 8 | RedihopÒ + Betacide as in test 1 + test 2 | | 30 + 10 | 15 | 2 | deionized water | 80 | 4 | 0 | 24 |
| 9 | deionized water | none | NA | NA | NA | none | 0 | 1 | 0 | 24 |
| 10 | Betacide | beta acids | 10 | NA | 4 | deionized water | 20 | 1 | ND | 24 |
| 11 | Redihopâ | rhoisoalpha acids | 30 | 30 | NA | deionized water | 13 | 1 | ND | 24 |
| 12 | RedihopÒ + Betacide as in test 1 + test 2 | | 30 + 10 | 15 | 2 | deionized water | 13 | 1 | ND | 24 |
| 13 | IsohopÒ | isoalpha acids | 30 | 30 | NA | deionized water | 70 | 1 | 33 | 24 |
| 14 | Tetrahop Goldâ | tetrahydroisoalpha acids | 9 | 9 | NA | deionized water | 81 | 1 | 0 | 24 |
| 15 | Hexahop GoldÒ | hexahydroisoalpha acids plus | 5 | 5 | NA | deionized water | 100 | 1 | 7 | 24 |
| | | tetrahydroisoalpha acids | 5 | 5 | NA | | | | | |
| 16 | corn starch | none | NA | NA | NA | none | 13 | 5 | ND | ND |
| 17 | MgBeta | magnesium salt of beta acids | 59.5 | NA | 4.3 | corn starch | 38 | 5 | ND | ND |
| 18 | MgBeta | magnesium salt of beta acids | 59.5 | NA | 8.5 | corn starch | 67 | 5 | 0 | 24 |
| 19 | MgRIAA | magnesium salt of rhoisoalpha | 65.5 | 65.5 | NA | corn starch | 7 | 5 | ND | ND |
| | | acids | | | | | | | | |
| 20 | corn starch | none | NA | NA | NA | none | 17 | 24 | 0 | 24 |
| 21 | MgTetrahop Gold | mg salt of tetrahydroisoalpha | 75.8 | 25.3 | NA | corn starch | 50 | 24 | 0 | 24 |
| | | acids | | | | | | | | |
| 22 | MgHexahop Gold | mg salt of hexahydroisoalpha | 36.7 | 12.2 | NA | corn starch | 50 | 24 | 0 | 24 |
| | | acids | | | | | | | | |
| | | plus mg salt of tetrahydroiso- | 36.7 | 12.2 | NA | | | | | |
| | | alpha acids | | | | | | | | |

NA means Not Applicable,
ND means No Data

### Miticide Screening Results

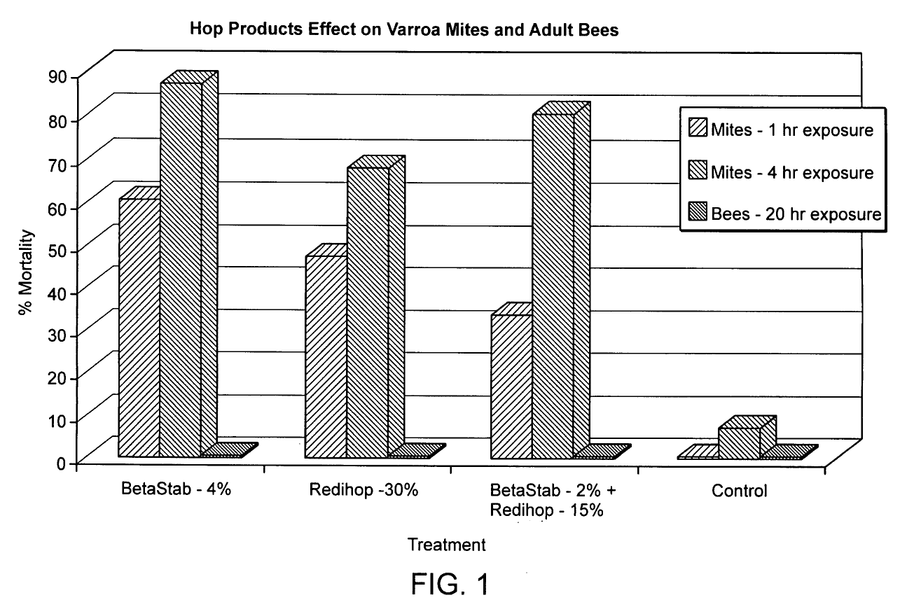

Results for the tests described in Table 2 are shown in FIG. 1.

In Tests 1-4 after four hours exposure, 5% beta acids killed 73% of Varroa mites; 15% rhoisoalpha acids killed 21% of Varroa mites; and a combination of 2.5% beta acids/7.5% rhoisoalpha acids produced 43% mortality of mites. Under control conditions only 7% mite mortality was observed. The majority of adult bees survived exposure to these same concentrations of alpha and beta acids. Specifically, 100% adult bees survived exposure to rhoisoalpha acids; 80% of adult bees survived exposure to 5% beta acids; and 93% of adult bees survived exposure to the combination of 2.5% beta acids/7.5% alpha acids. These results are presented in Table 2.

In Tests 5-8 following four hours of exposure, 4% beta acids killed 87% of Varroa mites; 30% rhoisoalpha acids killed 68% of mites; and the combination of 15% rhoisoalpha acids and 2% beta acids killed 80% of mites. 7% mite mortality was observed under control conditions. Adult bees exposed to these same product concentrations for 24 hours showed 100% survival. These results are presented in Table 2.

In Tests 9-15 after one hour of exposure, 4% beta acids killed 20% of Varroa mites; 30% rhoisoalpha acids killed 13% of mites; the combination of 15% rhoisoalpha acids and 2% beta acids killed 13% of mites; 9% tetrahydroisoalpha acids killed 81% of mites; the combination of 5% tetrahydroisoalpha acids and 5% hexahydroisoalpha acids killed 100% of mites. No mite mortality was observed under control conditions. Adult bees exposed to these product concentrations for 24 hours showed 67% survival after exposure to isoalpha acids; 93% survival after exposure to the combination of 5% tetrahydroisoalpha acids and 5% hexahydroisoalpha acids; 100% survival after exposure to 9% tetrahydroisoalpha acids; and 100% survival after exposure to control conditions. These results are presented in Table 2.

In Tests 16-19, after five hours of exposure, 8.5% beta acids in the form of a magnesium salt killed 67% of Varroa mites; 65.45% rhoisoalpha acids in the form of a magnesium salt killed 7% of Varroa mites. 13% of mites died under control conditions. 100% of bees survived after 24 hours exposure to 8.5% beta acids as a magnesium salt. These results are presented in Table 2.

In Tests 20-22 after 24 hours of exposure, 25.27% tetrahydroisoalpha acids in the form of the magnesium salt killed 50% of Varroa mites; and a combination of 12.23% tetrahydroisoalpha acids and 12.23% hexahydroisoalpha acids both in the form of magnesium salt killed 50% kill of Varroa mites. 17% kill of mites died under control conditions. 100% of adult honey bees survived for 24 hours under the same conditions. These results are presented in Table 2.

### Example 5

### Lepidoptera Control

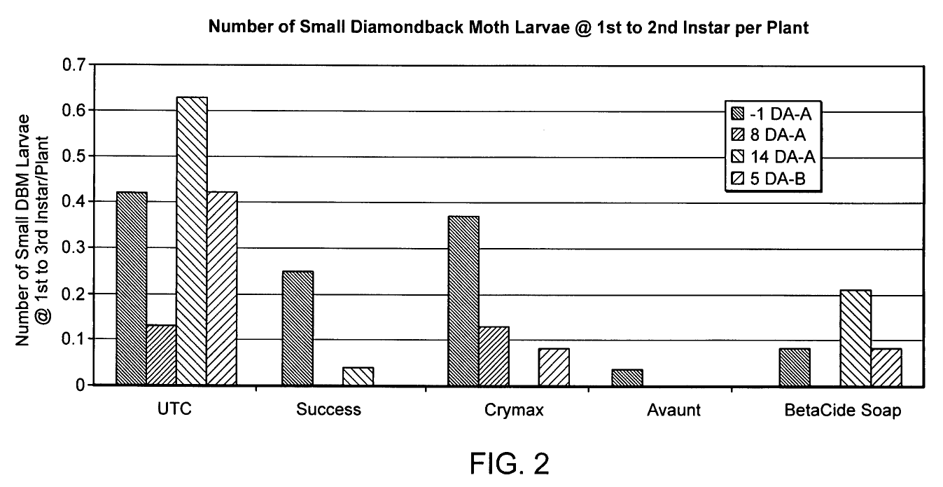

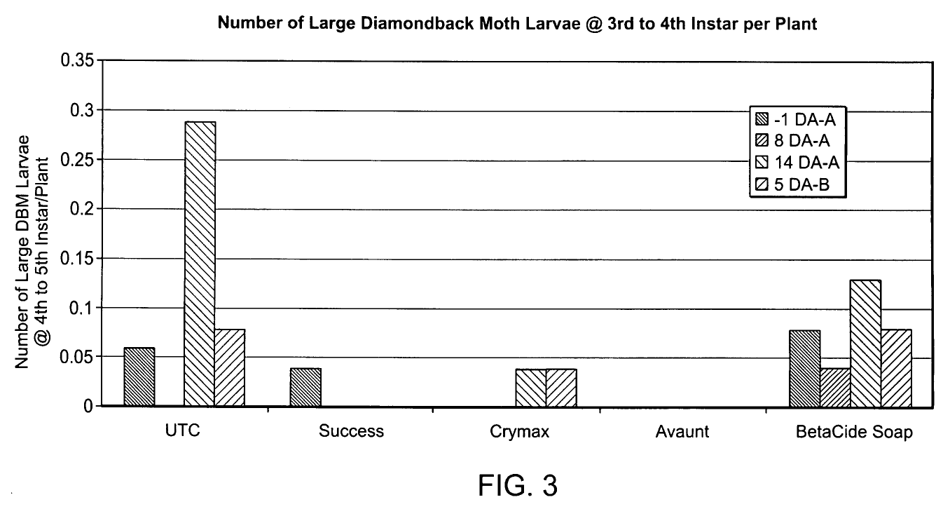

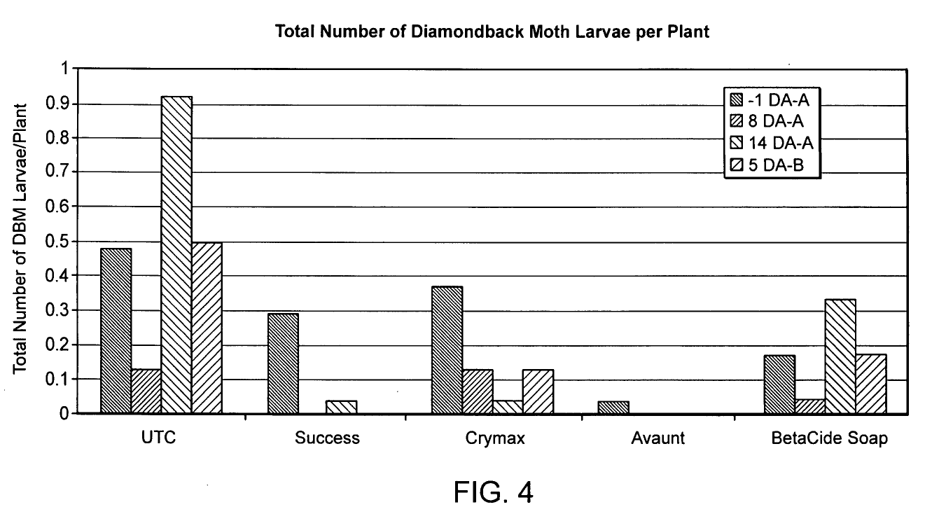

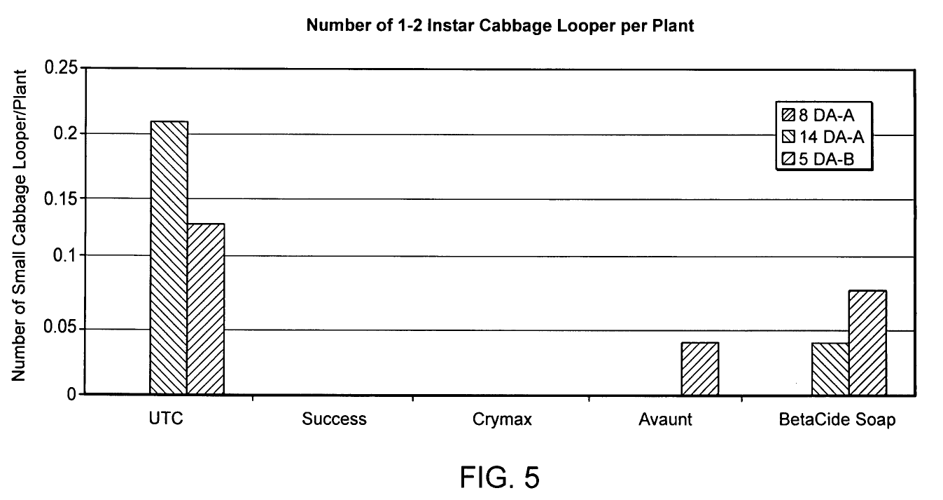

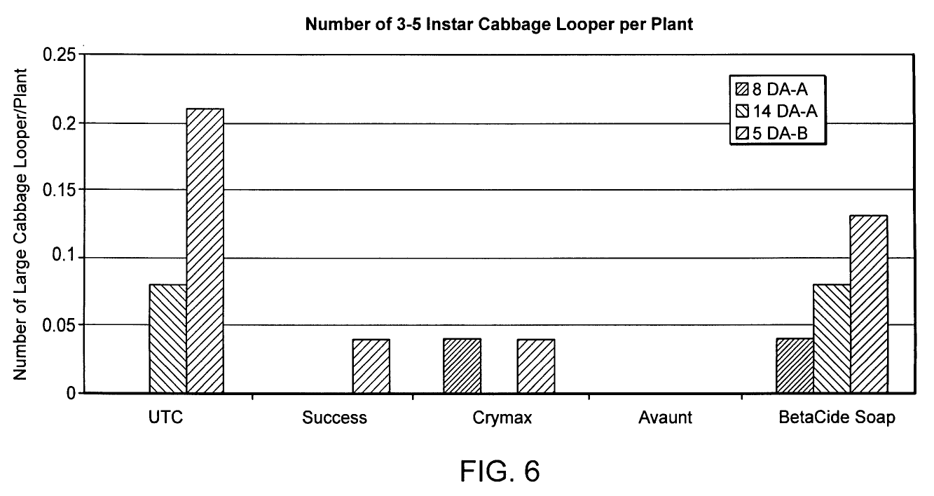

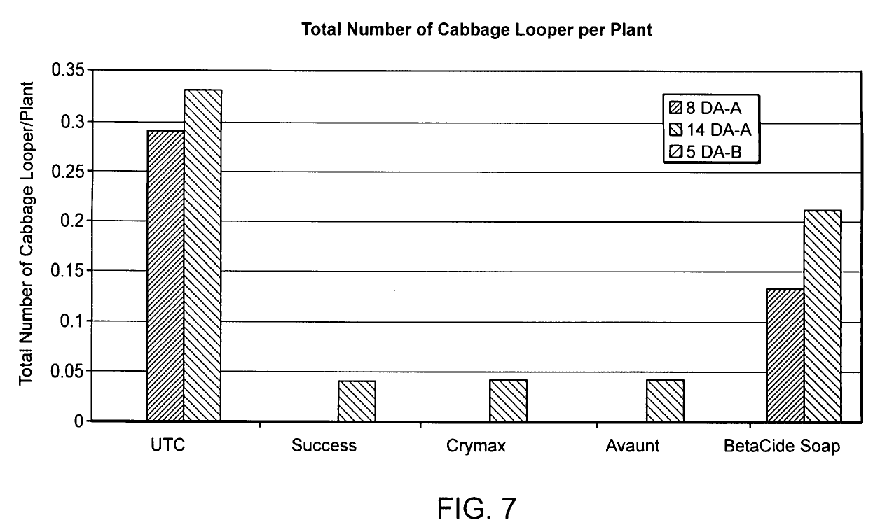

BetaCide was evaluated for the control of several Lepidoptera pest species on broccoli in the summer of 2005 in Santa Maria Calif. During this season, larval populations were sporadic. Therefore, foliar evaluations of larval feeding following the application period was used to examine whether Betacide was effective in controlling Lepidoptera. Insecticides were applied to each plot two times at 15 day intervals over the course of four weeks. The following formulations were used: Success at 5 fl oz/a; Crymax at 2 lb/a; BetaCide at 10% v/v with Soap at 0.5% v/v; Avaunt at 3.43 oz/a. The soap used was a liquid handsoap (Renown Pink handsoap, Deerfield, Ill.). Treatment applications were performed using a CO₂ backpack sprayer. The spray boom incorporated six D4 nozzles with #25 spinners and was operated at a pressure of 40-50 psi. Treatments were applied at a dilution of 75 GPA. The boom size was adjusted according to the growth stage of plant to ensure a thorough foliar coverage. Evaluation consisted of identifying and counting Lepidoptera pests on six randomly selected plants per plot. The plot dimensions were 3.33′×15′; row spacing was 3.33′; plant spacing was 12″. The following pests were identified: Diamondback moth (Plutella xylostella), Cabbage Looper (Trichoplusia ni) and Imported Cabbage Worm (Pieris rapae). Results were compared to an untreated control plot. Results of these studies are shown in FIGS. 2-7. These results indicate that BetaCide controls Lepidopteran pests.

### Example 6

### American Foul Brood Minimum Inhibitory Concentration in Na-Betacid Dilutions

For each of Examples 6-9, K-Beta acids at 10% stock and Na-Beta acids at 9% stock were tested against honeybee pathogen strains: American Foul Brood (AFB) and European Foul Brood (EFB). Both strains were obtained from the ATCC Institute and the inoculum for each culture was standardized for each treatment using McFarland standards. The plates were inoculated with a sterile swab creating a “bacteria lawn” with an average of 300×10⁶/ml spores which is equivalent to McFarland 1. Experimental design, protocol development and proper dilutions of the products were made. The trials were performed three times for each microorganism in order to achieve accurate results.

A Na Beta stock solution was prepared containing 0.34% active ingredient or 3400 ppm. The stock solution was diluted as follows:
3.4×10⁻⁴ (340 ppm)
3.4×10⁻⁵ (34 ppm)
3.4×10⁻⁶ (3.4 ppm)
3.4×10⁻⁷ (0.34 ppm)
3.4×10⁻⁸ (0.034 ppm)
3.4×10⁻⁹ (0.0034 ppm)
   A set of 3 plates of each dilution, including 2 control plates, were inoculated with AFB spores. All plates were incubated for 72 hours, and then observations were made.

Growth of AFB was seen at the lowest three concentrations of: 3.4×10⁻⁷ (0.34 ppm), 3.4×10⁻⁸ (0.034 ppm) and 3.4×10⁻⁹ (0.0034 ppm) in the inoculated Petri dishes. In addition, the presence of AFB was confirmed by Gram stain. No AFB were observed at 3.4×10⁻⁶ (3.4 ppm). This was confirmed by Gram stain. Thus, the MIC was determined to be 3.4 ppm.

### Example 7

### MIC for AFB in K-Betacid Dilutions

A K Beta Stock solution containing 10% beta acids or (100,000 ppm) was used for this test. The stock solution was diluted as follows:
1×10⁻² (10,000 ppm)
1×10⁻³ (1,000 ppm)
1×10⁻⁴ (100 ppm)
1×10⁻⁵ (10 ppm)
1×10⁻⁶ (1.0 ppm)
1×10⁻⁷ (0.1 ppm)
1×10⁻⁸ (0.01 ppm)
   A set of three plates of each dilution, including 2 control plates, were inoculated with AFB spores. All plates were allowed to incubate for 72 hours, and then observations were made. Growth of AFB was seen at the lowest three concentrations of: 1×10⁻⁶ (1.0 ppm); 1×10⁻⁷ (0.1 ppm) and 1×10⁻⁸ (0.01 ppm) in the inoculated Petri dishes. In addition, the presence of AFB was confirmed by Gram stain. No growth of AFB was observed at 1×10⁻⁵ (10 ppm). This was confirmed by Gram stain as well. The MIC was determined to be 10 ppm.

### Example 8

### European Foul Brood MIC in Na-Betacid Dilutions

A Na Beta stock solution was prepared containing 0.34% beta acids or 3400 ppm. The stock solution was diluted as follows:
3.4×10⁻⁴ (340 ppm)
3.4×10⁻⁵ (34 ppm)
3.4×10⁻⁶ (3.4 ppm)
3.4×10⁻⁷ (0.34 ppm)
3.4×10⁻⁸ (0.034 ppm)
3.4×10⁻⁹ (0.0034 ppm)
   A set of 3 plates of each dilution, including 2 control plates were inoculated with EFB spores. All plates were allowed to incubate for 72 hours under anaerobic conditions and observations were made. Growth of EFB was seen at the lowest three concentrations of: 3.4×10⁻⁷ (0.34 ppm); 3.4×10⁻⁸ (0.034 ppm) and 3.4×10⁻⁹ (0.0034 ppm) in the inoculated Petri dishes. No EFB growth was observed at 3.4×10⁻⁶ (3.4 ppm). The MIC was determined to be 3.4 ppm.

### Example 9

### EFB Growth and MIC in K-Betacid Dilutions

A K Beta Stock solution contained 10% active ingredient or (100,000 ppm) was used for the test. The stock solution was diluted as follows:
1×10⁻² (10,000 ppm)
1×10⁻³ (1,000 ppm)
1×10⁻⁴ (100 ppm)
1×10⁻⁵ (10 ppm)
1×10⁻⁶ (1.0 ppm)
1×10⁻⁷ (0.1 ppm)
1×10⁻⁸ (0.01 ppm)
   A set of 3 plates of each dilution, including 2 control plates were inoculated with EFB spores. All plates were allowed to incubate for 72 hours under anaerobic conditions and observations were made. Growth of EFB was seen at the lowest three concentrations of: 1×10⁻⁶ (1.0 ppm); 1×10⁻⁷ (0.1 ppm) and 1×10⁻⁸ (0.01 ppm) in the inoculated Petri dishes. No EFB growth was observed at 1×10⁻⁵ (10 ppm). The MIC was determined to be 10 ppm.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or sub combination) of listed elements. The recitation of an embodiment herein includes that embodiment as any single embodiment or in combination with any other embodiments or portions thereof.

All references herein are incorporated by reference in their entirety. All patents and publications mentioned in this specification are herein incorporated by reference to the same extent as if each independent patent and publication was specifically and individually indicated to be incorporated by reference.

What is claimed is:

1. A method of inhibiting a honey bee pathogen, the method comprising contacting the pathogen with an effective amount of a composition comprising a hop derivative, thereby controlling a honey bee pathogen.

2. The method of claim 1, wherein the pathogen is a bacterial or fungal pathogen.

3. The method of claim 2, wherein the bacterial pathogen is Melissococcus plutonis or Paenibacillus larvae and the fungal pathogen is Ascosphaera apis.

4. (canceled)

5. A method of treating or preventing a pathogen infection in a honey bee hive, the method comprising contacting the hive with an effective amount of a composition comprising a hop derivative, thereby treating or preventing a pathogen infection in a honey bee hive.

6. The method of claim 5, wherein the pathogen infection is a bacterial infection.

7. The method of claim 6, wherein the bacterial pathogen is Melissococcus plutonis or Paenibacillus larvae.

8. The method of claim 5, wherein the infection is American or European foulbrood.

9. The method of claim 5, wherein the pathogen is a fungal pathogen.

10. The method of claim 5, wherein the fungal pathogen is Ascosphaera apis.

11. The method of claim 1 or 5, wherein the hop derivative is an alpha acid or beta acid.

12-14. (canceled)

15. The method of claim 1 or 5, wherein the composition comprises a combination of alpha and beta acids.

16-18. (canceled)

19. A method of treating or preventing a Lepidoptera infestation in a honey bee hive, the method comprising contacting the hive with an effective amount of a composition comprising a hop derivative, thereby treating or preventing a Lepidoptera infestation in a honey bee hive.

20. The method of claim 19, wherein the Lepidoptera is the wax moth, Galleria mellonella.

21. (canceled)

22. A method for maintaining the health of a honey bee hive, the method comprising:
contacting the hive with an effective amount of a composition comprising a hop derivative, thereby maintaining the health of the honey bee hive.

23-24. (canceled)

25. The method of claim 1 or 22, wherein the composition comprises a sodium, potassium, or magnesium salt of alpha or beta acids.

26-27. (canceled)

28. A composition for treating or preventing a honey bee pathogen infection and/or hive infestation, the composition comprising an effective amount of a hop derivative in a suitable form for delivery to a bee or hive.

29. The composition of claim 28, wherein the effective amount inhibits the growth, proliferation, or survival of a bacterial or fungal pathogen.

30. The composition of claim 28, wherein the effective amount controls an acarid or destructive insect.

31. The composition of claim 28, wherein the hop derivative is an alpha acid, beta acid, or combination of an alpha and a beta acid.

32-39. (canceled)

40. The composition of claim 28, wherein the form is selected from the group consisting of a liquid, a powder, an oil, an emulsion, a capsule, and a vapor.

41-44. (canceled)

45. A controlled release composition for treating or preventing a composition for treating or preventing a honey bee pathogen infection and/or hive infestation, the composition comprising an effective amount of a hop derivative in a suitable form for controlled-release delivery to a honey bee or honey bee hive.

46-49. (canceled)

50. The composition of claim 45, wherein the pathogen infection is a Melissococcus plutonis or Paenibacillus larvae infection.

51. The composition of claim 50, wherein the composition treats or prevents American foulbrood, European foulbrood, or chalkbrood.

52. (canceled)

53. The composition of claim 45, wherein the hive infestation is a Varroa mite or wax moth infestation.

54. The composition of claim 45, wherein the composition comprises a sodium, potassium, or magnesium salt of alpha or beta acids.

55-56. (canceled)

57. A biocide delivery device, the device comprising a composition of claim 28 or 45.

58. The biocide delivery device of claim 57, wherein the device is selected from the group consisting of a strip, controlled release strip, tablet, reservoir, polymer disc, evaporation device, fiber, tube, polymeric block, membrane, pellet, and microcapillary.

59. A hive comprising a composition of claim 28 or 45.

60-61. (canceled)

62. A kit for the treatment or prevention of a pathogen infection or hive infestation, the kit comprising an effective amount of a hop derivative in a form suitable for delivery to a site of infection or infestation.

63-64. (canceled)

65. A method of identifying a hop derivative that inhibits a bacterial or fungal pathogen, the method comprising
(a) contacting a bacterial or fungal culture with a test composition comprising a hop derivative; and
(b) assaying bacterial or fungal growth.

66-73. (canceled)

74. A method of identifying a hop derivative that disrupts a biological function of a wax moth, the method comprising
(a) contacting the wax moth with a test composition comprising a hop derivative; and
(b) assaying the wax moth biological function.

75-81. (canceled)
